SEQUENCE LISTING (16 sequences)

SEQ ID NO: 1 - protein, 42 aa, synthetic construct
  source 1..42: note = amino acid sequence of the TMD of hCD137 introducing the cysteine mutation
CIISFFLALTSTALLFLLFFLTLRFSVVKRGRKKLLYIFKQP

SEQ ID NO: 2 - protein, 63 aa, synthetic construct
  source 1..63: note = amino acid sequence of the TMD of hHER2 introducing the cysteine mutation
CPAEQRASPLTSIISAVVGILLVVVLGVVFGILIKRRQQKIRKYTLRRLLQETELVEPLTPSG

SEQ ID NO: 3 - protein, 207 aa, synthetic construct
  source 1..207: note = amino acid sequence of MSP nanodisc after introducing the Strep-tag
MGSSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQGGGGSSSAWSHPQFE

SEQ ID NO: 4 - protein, 169 aa, synthetic construct
  source 1..169: note = amino acid sequence of the ECD peptide segment of hCD137 including a His tag
HHHHHHLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 5 - protein, 156 aa, synthetic construct
  source 1..156: note = amino acid sequence of Sortase A after a His tag is added to the carboxyl terminus of Sortase A
MQAKPQIPKDKSKVAGYIEIPDADIKEPVYPGPATREQLNRGVSFAEENESLDDQNISIAGHTFIDRPNYQFTNLKAAKKGSMVYFKVGNETRKYKMTSIRNVKPTAVEVLDEQKGKDKQLTLITCDDYNEETGVWETRKIFVATEVKLEHHHHHH

SEQ ID NO: 6 - protein, 43 aa, synthetic construct
  source 1..43: note = the TMD amino acid sequence containing the enzyme ligation site G of Sortase A and cysteine mutation
GCIISFFLALTSTALLFLLFFLTLRFSVVKRGRKKLLYIFKQP

SEQ ID NO: 7 - DNA, 129 bp, synthetic construct
  source 1..129: mol_type = other DNA; note = nucleotide sequence encoding the amino acid sequence of SEQ ID NO: 1
ggctgtattatatcatttttcctggcgctgacctccaccgctctgctgttcctcttgtttttcttgacgctgcgttttagcgttgtgaagcgcggtcgtaaaaagctgttatacatctttaaacagccg

SEQ ID NO: 8 - DNA, 627 bp, synthetic construct
  source 1..627: mol_type = other DNA; note = optimized nucleotide sequence encoding MSP
atgggcagcagcacctttagcaaactgcgtgaacagctgggcccggtgacccaggaattttgggataacctggaaaaagaaaccgaaggcctgcgtcaggaaatgagcaaagatctggaagaggtgaaagcgaaagtgcagccgtatctggatgactttcagaaaaaatggcaggaagagatggaactgtatcgtcagaaagtggaaccgctgcgtgcggaactgcaggaaggcgcgcgtcagaaactgcatgaactgcaggaaaaactgagcccgctgggcgaagagatgcgtgatcgtgcgcgtgcgcatgtggatgcgctgcgtacccatctggcgccgtatagcgatgaactgcgtcagcgtctggcggcccgtctggaagcgctgaaagaaaacggcggtgcgcgtctggcggaatatcatgcgaaagcgaccgaacatctgagcaccctgagcgaaaaagcgaaaccggcgctggaagatctgcgtcagggcctgctgccggtgctggaaagctttaaagtgagctttctgagcgcgctggaagagtataccaaaaaactgaacacccagggcggcggcggcagcagcagcgcgtggtcgcatccacagttcgagaaataa

SEQ ID NO: 9 - protein, 175 aa, synthetic construct
  source 1..175: note = amino acid sequence of the modified hCD137 ECD containing both the His tag and the enzyme ligation site of Sortase A
HHHHHHLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQLPETGG

SEQ ID NO: 10 - DNA, 525 bp, synthetic construct
  source 1..525: mol_type = other DNA; note = gene sequence of hCD137 ECD optimized to the codons preferred by HEK 293
caccaccaccaccaccacctgcaggacccctgctctaactgtcctgccggcaccttctgcgataacaatcggaaccagatctgctccccatgtccccctaattcttttagctccgccggcggccagaggacatgcgatatctgtcgccagtgcaagggcgtgttccggaccagaaaggagtgttctagcacaagcaacgccgagtgcgactgtacccctggatttcactgcctgggagcaggatgttccatgtgcgagcaggattgtaagcagggccaggagctgaccaagaagggctgcaaggactgctgtttcggcaccttcaacgatcagaagcggggcatctgtagaccatggaccaactgcagcctggacggcaagtccgtgctggtgaatggcacaaaggagagggacgtggtgtgcggaccttctccagcagatctgagcccaggagcatcctctgtgacaccaccagcaccagcaagagagcctggacactccccacagctgcctgagacaggcggc

SEQ ID NO: 11 - DNA, 468 bp, synthetic construct
  source 1..468: mol_type = other DNA; note = gene sequence of Sortase A optimized to the codons preferred by E. coli
atgcaagctaaacctcaaattccgaaagataaatcaaaagtggcaggctatattgaaattccagatgctgatattaaagaaccagtatatccaggaccagcaacacgcgaacaattaaatagaggtgtaagctttgcagaagaaaatgaatcactagatgatcaaaatatttcaattgcaggacacactttcattgaccgtccgaactatcaatttacaaatcttaaagcagccaaaaaaggtagtatggtgtactttaaagttggtaatgaaacacgtaagtataaaatgacaagtataagaaacgttaagccaacagctgtagaagttctagatgaacaaaaaggtaaagataaacaattaacattaattacttgtgatgattacaatgaagagacaggcgtttgggaaacacgtaaaatctttgtagctacagaagtcaaactcgagcaccaccaccaccaccac

SEQ ID NO: 12 - DNA, 405 bp, synthetic construct
  source 1..405: mol_type = other DNA; note = gene sequence of anti-hCD137 nanobody optimized to the codons preferred by E. coli
atgcaccaccaccaccaccacgaaaacctgtactttcagagcgaggtgcaactgctggaaagcggtggcggtgaggttcaaccgggcggtagcctgcgtctgagctgcgcggcgagcggtttcagctttagcatcaacgcgatgggctggtatcgtcaagcgccgggcaagcgtcgtgaattcgtggcggcgatcgagagcggccgtaacaccgtgtacgcggaaagcgttaagggtcgttttaccattagccgtgacaacgcgaaaaacaccgtgtatctgcagatgagcagcctgcgtgcggaggataccgcggtttactattgcggcctgctgaagggtaaccgtgtggttagcccgagcgttgcgtactggggccaaggtaccctggtgaccgttaaaccg

SEQ ID NO: 13 - protein, 69 aa, synthetic construct
  source 1..69: note = amino acid sequence of the TMD including a Sortase A ligation site and a cysteine mutation for conjugation
GGGGGCPAEQRASPLTSIISAVVGILLVVVLGVVFGILIKRRQQKIRKYTLRRLLQETELVEPLTPSGA

SEQ ID NO: 14 - protein, 173 aa, synthetic construct
  source 1..173: note = amino acid sequence of the hHER2 ECD with His tag and enzyme ligation site of Sortase A
HHHHHHHHHHGGGGSHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGLPETGG

SEQ ID NO: 15 - protein, 146 aa, synthetic construct
  source 1..146: note = sequence of the nanobody with a His tag and a TEV cleavage site added to the amino terminus of the nanobody
MGSAHHHHHHGGGSGGGSENLYFQSEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTLVTVSSGSC

SEQ ID NO: 16 - DNA, 441 bp, synthetic construct
  source 1..441: mol_type = other DNA; note = gene sequence of anti-hHER2 nanobody optimized to the codons preferred by Saccharomyces
atgggatcagctcaccaccatcaccaccacggtggtggctcgggcgggggctcggagaacctgtatttccagagcgaggttcaactggtggagagtggtggcggcctggtgcaagcgggtggttccctgagactgagctgcgcggcgagcggcattacctttagcatcaacacgatgggttggtaccgccaggctccgggtaaacagcgtgaacttgtcgcactcatcagctccatcggcgacacctattacgcagactccgtgaagggtcgtttcaccattagccgtgacaacgcgaaaaataccgtttacctgcagatgaatagcttgaagccggaagatactgcggtttactactgcaaacgttttcgcaccgctgcccaaggtacggattattggggtcagggtacgttggttaccgtgagctctggttcttgttaa

(19) United States
(12) Patent Application Publication
(10) Pub. No.: US 2024/0402188 A1
(43) Pub. Date: Dec. 5, 2024

(54) SITE-SPECIFICALLY LABELED TRANSMEMBRANE PROTEIN NANODISC, AND PREPARATION METHOD AND USE THEREOF

(71) Applicant: Nankai University, Tianjin (CN)

(72) Inventors: Chan CAO, Tianjin (CN); Junyuan GONG, Tianjin (CN); Xiaohui ZHOU, Tianjin (CN); Yifan LIN, Tianjin (CN); Jialu GENG, Tianjin (CN)

(21) Appl. No.: 18/676,905

(22) Filed: May 29, 2024

(30) Foreign Application Priority Data
May 31, 2023 (CN) 202310631483.4

(51) Int. Cl. G01N 33/68; C07K 14/705

(52) U.S. Cl. CPC G01N 33/6854; C07K 14/705

(57) ABSTRACT

The application belongs to the technical field of biotechnology, and specifically relates to a site-specifically labeled transmembrane protein nanodisc, and a preparation method and use thereof. The preparation method for a site-specifically labeled transmembrane domain (TMD) as provided in this disclosure involves introducing a cysteine mutation into the TMD gene of a transmembrane protein. This mutated TMD is then conjugated with a chemical compound to achieve site-specifical labeling. Subsequently, the site-specifically labeled TMD and an extracellular domain (ECD) are expressed separately. The TMD is incorporated into a nanodisc, which closely mimics the physiological environment, and then ligated to the ECD. This process results in the formation of a site-specifically labeled transmembrane protein nanodisc.

### CROSS-REFERENCE TO RELATED APPLICATIONS

This patent application claims the benefit and priority of Chinese Patent Application No. 202310631483.4, filed with the China National Intellectual Property Administration on May 31, 2023, the disclosure of which is incorporated by reference herein in its entirety as part of the present application.

### REFERENCE TO SEQUENCE LISTING

A computer readable XML file entitled “GWP20240100835_sequence listing”, which was created on May 21, 2024 with a file size of about 22,446 bytes, contains the sequence listing for this application, has been filed with this application, and is hereby incorporated by reference in its entirety.

### TECHNICAL FIELD

The present disclosure belongs to the technical field of biotechnology, and specifically relates to a site-specifically labeled transmembrane protein nanodisc, and a preparation method and use thereof.

### BACKGROUND OF THE INVENTION

A transmembrane protein refers to a protein embedded in the lipid bilayer, spanning the membrane. Cells can sense their surrounding environment through transmembrane proteins and respond to extracellular environment to achieve signal transduction, thereby regulating their state. Transmembrane proteins are involved in a variety of important physiological and pathological processes, including cell proliferation, cell differentiation, cell survival, apoptosis, angiogenesis, and tumorigenesis. Many diseases are related to dysfunction of transmembrane proteins, such as diabetes mellitus caused by abnormal insulin receptor function, and tumors caused by dysregulation of human epidermal growth factor receptor-2 (HER2 receptor).

The function of transmembrane proteins makes them the targets of many drugs. For instance, G protein-conjugated receptors (GPCRs), which constitute about 30% of current drug targets. Since transmembrane proteins, which span the lipid bilayer entirely, have highly hydrophobic transmembrane domains (TMDs) that promote aggregation into insoluble precipitates in aqueous solutions. Therefore, preserving the native structure and activity of transmembrane proteins for in vitro studies requires reconstituting them into membrane-mimetic environments.

Current methods for preparing transmembrane proteins in vitro involve expressing full-length antigen in cells followed by direct extraction and purification using detergents. However, this approach presents several challenges including low expression yields, low extraction yields, heterogeneity, and difficulty in site-specific labeling.

### SUMMARY OF THE INVENTION

An objective of the present disclosure is to provide a site-specifically labeled transmembrane protein nanodiscs, and a preparation method and use thereof. The preparation methods enable site-specific labeling of transmembrane proteins and increasing their final yields.

The present disclosure provides a method for preparing a site-specifically labeled TMD, including the following steps:
Mutating a TMD-encoding gene to introduce a cysteine residue, resulting in a gene fragment of the transmembrane region containing cysteine mutations;
Expressing the modified gene to obtain a TMD peptide segment with the cysteine site; and
conjugating a chemical compound to the cysteine site of the TMD peptide segment, resulting in a TMD with a site-specific label.

Preferably, the transmembrane protein includes transmembrane proteins containing extracellular domain (ECD) and transmembrane domain (TMD).

The present disclosure further provides a site-specifically labeled TMD prepared by the preparation method.

The present disclosure further provides a site-specifically labeled TMD nanodisc with a site-specifically labeled site in the transmembrane region, which includes the site-specifically labeled TMD and a nanodisc as described in the above technical solution; the nanodisc encapsulates or is inserted into the TMD with a site-specifically labeled site.

Preferably, the nanodisc is composed of phospholipids.

The present disclosure further provides a site-specifically labeled transmembrane protein nanodisc, including the site-specific labeled TMD and an ECD.

The present disclosure further provides a method for preparing a full-length transmembrane protein nanodisc by ligating the amino terminus of the site-specifically labeled TMD nanodisc to the carboxyl terminus of the ECD.

Preferably, the ligation is performed using enzymatic or peptide bond formation methods.

Preferably, the enzyme used for ligation is either Sortase or Butelase.

The present disclosure further provides use of the site-specifically labeled TMD, the site-specifically labeled TMD nanodisc, the site-specifically labeled transmembrane protein nanodisc, or a site-specifically labeled transmembrane protein nanodisc prepared by the preparation method in antibody screening and/or verification.

### Beneficial Effects:

The present disclosure provides a method for preparing a site-specifically labeled TMD by introducing a cysteine mutation into the TMD of a transmembrane protein and then conjugating with a chemical compound, resulting in a precisely labeled TMD.

The disclosed method separates the preparation of site-specifically labeled TMD from the ECD. The TMD is reconstituted into a nanodisc to provide a near-native lipid bilayer environment, and then ligated to the ECD, resulting in a site-specifically labeled full-length transmembrane protein nanodisc. Compared with traditional methods that involve expressing entire antigens, followed by detergent extraction and nanodisc reconstitution, this disclosed method enhances the preparation of transmembrane protein nanodisc by enabling site-specific labeling of the transmembrane domain, increasing yields of nanodiscs, and preserving ECD activity with a detergent-free process.

### BRIEF DESCRIPTION OF DRAWINGS

To illustrate the examples of the present disclosure or the technical solutions in the prior art more clearly, the accompanying drawings required in the examples will be briefly introduced below.

FIG. 1 shows the chromatographic purification profile for the hCD137 TMD prepared in Example 1;

FIG. 2A-2B show the identification of hCD137 TMD with cysteine insertion by SDS-PAGE gel (FIG. 2A) and mass spectrometry (MS) (FIG. 2B);

FIG. 3 shows the purification of hCD137 TMD-Proxyl by HPLC;

FIG. 4 shows the mass spectrometry of the hCD137-TMD-Proxyl in Example 2;

FIG. 5 shows the purification of MSP in Example 3, and the product was verified by SDS-PAGE gel;

FIG. 6 shows the process of Proxyl-TMD nanodisc reconstitution by SDS-PAGE gel;

FIG. 7 shows the purification results of hCD137 ECD by SDS-PAGE gel;

FIG. 8 shows the purification results of Sortase A by SDS-PAGE gel;

FIG. 9 shows the ligation results of nanodisc containing hCD137 ECD-TM by SDS-PAGE gel;

FIG. 10 shows the 19F-NMR spectrum of hCD137 ECD-TM nanodisc labeled with trifluoromethyl in Example 5;

FIG. 11 shows the purification results of the anti-hCD137 nanobody in Use Example 1;

FIG. 12 shows the co-elution of hCD137 transmembrane antigen nanodisc with nanobody by FPLC and SDS-PAGE gel;

FIG. 13 shows the mass spectrometry of the Proxyl-hHER2 TMD in Example 6;

FIG. 14 shows the process of Proxyl-hHER2 TMD nanodisc reconstitution by SDS-PAGE gel in Example 6;

FIG. 15 shows the detection results of hHER2 ECD by SDS-PAGE gel in Example 6;

FIG. 16 shows the detection results of the nanodisc incorporating hHER2 ECD^{IV}-TM by SDS-PAGE gel in Example 6;

FIG. 17 shows the detection gel results of the anti-hHER2 nanobody by SDS-PAGE gel in Use Example 2; and

FIG. 18 shows the co-elution of nanodisc incorporating hHER2 ECD^{IV}-TM with bound nanobody by FPLC and SDS-PAGE gel in Use Example 2.

### DETAILED DESCRIPTION OF THE INVENTION

The present disclosure provides a preparation method of a site-specifically labeled TMD, including the following steps:
introducing a cysteine mutation into a TMD gene fragment encoding a transmembrane protein to obtain a cysteine mutation-containing TMD gene fragment;
expressing the cysteine mutation-containing TMD gene fragment to obtain a cysteine mutation site-containing TMD peptide segment; and
conjugating a chemical compound to a cysteine mutation site on the cysteine mutation site-containing TMD peptide segment to obtain the site-specifically labeled TMD.

In the present disclosure, the transmembrane protein preferably includes but is not limited to a transmembrane protein containing ECD and TMD, further preferably includes CD137 or HER2, and more preferably includes hCD137 or hHER2. Taking hCD137 or hHER2 as an example, the preparation method of the present disclosure is preferably specifically preferred and limited, but it cannot be regarded as the entire protection scope of the present disclosure.

In the present disclosure, a cysteine mutation is introduced into a TMD gene fragment encoding a transmembrane domain to obtain a cysteine mutation-containing TMD gene fragment. A process of introducing the cysteine mutation preferably includes inserting a codon of cysteine at any position of the TMD gene fragment encoding the transmembrane protein.

In the present disclosure, the cysteine mutation-containing TMD gene fragment is expressed to obtain a cysteine mutation site-containing TMD peptide segment. Before conducting the expression, it is preferable to conduct codon optimization on the TMD gene fragment encoding the cysteine mutation according to the codon preference of a host, so as to achieve efficient expression of the transmembrane protein.

In the present disclosure, there is no special limitation on an expression method, and methods well known to those skilled in the art can be used, such as prokaryotic expression or eukaryotic expression. In an example, the TMD gene fragment encoding the hCD137 introducing the cysteine mutation is subcloned into a plasmid for the expression; the expression method is preferably prokaryotic expression; a host used is preferably E. coli and a plasmid preferably includes pMM-LR6. In an example, an amino acid sequence of the TMD of hCD137 introducing the cysteine mutation is preferably shown in SEQ ID NO: 1, specifically: CIISFFLALTSTALLFLLFFLTLRFSVVKRGRKKLLYIFKQP, and the cysteine mutation is located at an amino terminus of the TMD of hCD137. In another example, an amino acid sequence of the TMD of hHER2 introducing the cysteine mutation is preferably shown in SEQ ID NO: 2, specifically: CPAEQRASPLTSIISAVVGILLVVVLGVVFGILIKRRQQKIRKYTLRRLLQETEL VE PLTPSG, and the cysteine mutation is preferably located at a 642nd amino acid of hHER2.

In the present disclosure, a chemical compound is conjugated to a cysteine mutation site on the cysteine mutation site-containing TMD peptide segment to obtain the site-specifically labeled TMD. The chemical compound preferably includes a type of chemical compound conducting a specific reaction with cysteine, more preferably a free radical label, a dye, or a ¹⁹F-tag, such as a free radical label containing a maleimide reactive group, Cy3 or Cy5 dyes, ¹⁹F-chemical tag containing phenylsulfone substituents; the specific reaction preferably includes Michael addition, thiol substitution, or thiol cyclization. There are no special limitations on specific types of the free radical label, dye, and ¹⁹F-tag, and any type of the chemical compound can be used in the present disclosure. There is no special limitation on a conjugation method well known to those skilled in the art can be used. As in an example, a cysteine site on the TMD of hCD137 is preferably conjugated with 3-maleimide-2,2,5,5-tetramethylpyrrolidine-1-oxyl radical (3-Maleimido-Proxyl). Specifically, a powdered TMD peptide containing the cysteine mutation site is dissolved in a HEPES reaction buffer, and then was reacted with TCEP and 3-Maleimido-Proxyl to obtain a TMD containing Proxyl site-specifically labeled hCD137. The powdered TMD peptide containing the cysteine mutation site, the TCEP, and the 3-Maleimido-Proxyl are at a molar concentration ratio in the HEPES reaction buffer of preferably 1:2:2.

The present disclosure further provides a site-specifically labeled TMD prepared by the preparation method. In the present disclosure, site-specific labeling of the TMD of the transmembrane protein is achieved by introducing a cysteine mutation into the TMD of the transmembrane protein and then conjugating the cysteine mutation with a chemical compound.

The present disclosure further provides a site-specifically labeled TMD nanodisc, including the site-specifically labeled TMD and a nanodisc; where the nanodisc is wrapped on or inserted into the site-specifically labeled TMD.

In the present disclosure, the site-specifically labeled TMD nanodisc is preferably obtained by combining the site-specific label TMD with a nanodisc. The nanodisc preferably includes a phospholipid nanodisc, further preferably includes a membrane scaffold protein nanodisc (MSP nanodisc), a polypeptide scaffold nanodisc, or a synthetic nanodisc, more preferably the MSP nanodisc.

In the present disclosure, the nanodisc is preferably prepared by prokaryotic expression or eukaryotic expression. Before the prokaryotic expression or eukaryotic expression, a gene sequence of the nanodisc is preferably codon-optimized to enable efficient expression of the nanodisc. There are no special limitations on specific steps of prokaryotic expression or eukaryotic expression, and steps well known to those skilled in the art can be used. During the prokaryotic expression or eukaryotic expression, a purification tag is preferably introduced at the carboxyl terminus of the nanodisc for purification of the nanodisc. There is no particular limitation on a specific purification process, and those skilled in the art can make routine adjustments according to the corresponding transmembrane protein. In an example, the MSP nanodisc is preferably used, and a Strep-tag is introduced at the carboxyl terminus of the MSP nanodisc; the MSP nanodisc after introducing the Strep-tag has an amino acid sequence preferably shown in SEQ ID NO: 3, specifically:

MGSSTFSKLREQLGPVTQEFWDNLEKETEGLRQEMSKDLEEVKAKVQPYL
DDFQKKWQEEMELYRQKVEPLRAELQEGARQKLHELQEKLSPLGEEMRDR
ARAHVDALRTHLAPYSDELRQRLAARLEALKENGGARLAEYHAKATEHLS
TLSEKAKPALEDLRQGLLPVLESFKVSFLSALEEYTKKLNTQGGGGSSSA
WSHPQFE.

In the example, the MSP nanodisc is preferably prepared by prokaryotic expression, the host used is preferably E. coli, and the plasmid preferably includes pET-28b (+). In the example, the MSP nanodisc is preferably purified using a Streptactin affinity column to obtain a purified MSP nanodisc.

In the present disclosure, after the purified nanodisc is obtained, the site-specifically labeled TMD is preferably combined with the purified MSP nanodisc through Bicelle reconstitution to obtain a site-specifically labeling TMD nanodisc. In an example, a specific process of combining the TMD containing Proxyl site-specifically labeled hCD137 with the purified MSP nanodisc preferably includes: dissolving the powdered TMD containing Proxyl site-specifically labeled hCD137 in urea, mixing with DMPC and DHPC, drying a resulting mixture under N₂ environment and then lyophilized to obtain a powdered mixture; dissolve the powdered mixture with a urea buffer to obtain a urea solution; dialyzing the mixture solution against HEPES buffer to allow bicelle reconstitution, mixing bicelles with the purified MSP nanodisc, and continuing dialysis to obtain the nanodisc with the TMD containing Proxyl site-specifically labeled hCD137. In the example, the TMD containing Proxyl site-specifically labeled hCD137, the DMPC, and the DHPC are at a concentration ratio of preferably 1:35:140. The urea buffer preferably includes components at the following concentrations: 8 M urea, 20 mM HEPES, and 75 mM NaCl; and the urea buffer has a pH value of preferably 7.4. The HEPES buffer preferably includes components at the following concentrations: 20 mM HEPES and 75 mM NaCl, and has a pH value of preferably 7.4. A dialysis bag for dialysis has a molecular weight cut-off of preferably 3 kDa.

The present disclosure further provides a site-specifically labeled transmembrane protein nanodisc, including the site-specifically labelled TMD nanodisc and an ECD.

The present disclosure further provides a preparation method of the site-specifically labeled transmembrane protein nanodisc, including the following steps: ligating an amino terminus of the site-specifically labeled TMD nanodisc to a carboxyl terminus of the ECD to obtain the site-specifically labeled transmembrane protein nanodisc.

In the present disclosure, the ECD peptide segment is preferably prepared; a preparation method specifically preferably includes: expressing a gene fragment encoding the ECD peptide segment to obtain the ECD peptide segment. The expression preferably includes prokaryotic expression or eukaryotic expression, more preferably the eukaryotic expression. Before the expression, the gene fragment encoding the ECD peptide segment is preferably codon optimized to enable efficient expression of the ECD peptide segment. During the expression, a purification tag is preferably introduced at the amino terminus of the ECD peptide segment, thereby purifying the ECD peptide segment. In an example, the gene fragment encoding the ECD peptide of hCD137 is preferably codon-optimized and subcloned into a pcDNA3.4 (+) plasmid, and a resulting expression vector is transiently transfected to obtain the ECD peptide segment of hCD137. The ECD peptide segment of hCD137 preferably includes a His tag, and has an amino acid sequence preferably shown in SEQ ID NO: 4, specifically:

HHHHHHLQDPCSNCPAGTFCDNNRNQICSPCPPNSFSSAGGQRTCDICRQ
CKGVFRTRKECSSTSNAECDCTPGFHCLGAGCSMCEQDCKQGQELTKKGC
KDCCFGTFNDQKRGICRPWTNCSLDGKSVLVNGTKERDVVCGPSPADLSP
GASSVTPPAPAREPGHSPQ.

In the present disclosure, after obtaining the ECD peptide segment, the site-specifically labeled TMD nanodisc is ligated to the ECD peptide segment to obtain the site-specifically labeled transmembrane protein nanodisc. The ligation preferably includes enzyme ligation or peptide chain ligation; the enzyme ligation is preferably enzyme-mediated peptide chain ligation, and the peptide chain ligation is preferably chemical small molecule-mediated peptide chain ligation; an enzyme used in the enzyme ligation preferably includes a Sortase or a Butelase, more preferably Sortase A. There is no particular limitation on specific steps of the enzyme ligation, and the steps can be adjusted routinely according to the transmembrane protein and the enzyme used.

In the present disclosure, when the ligation is conducted by the enzyme ligation, enzyme ligation sites are introduced at the amino terminus of the TMD and the carboxyl terminus of the ECD of the transmembrane protein, respectively. As in an example, it is preferable to introduce an enzyme connection site G of Sortase A at the amino terminus of the TMD of hCD137, and to introduce an enzyme ligation site LPETGG of the Sortase A at the carboxyl terminus of the ECD of hCD137. Preferably, a His tag is further added to the carboxyl terminus of the Sortase A to allow protein purification. After adding the His tag, there is an amino acid sequence preferably shown in SEQ ID NO: 5, specifically:

MQAKPQIPKDKSKVAGYIEIPDADIKEPVYPGPATREQLNRGVSFAEENE
SLDDQNISIAGHTFIDRPNYQFTNLKAAKKGSMVYFKVGNETRKYKMTSI
RNVKPTAVEVLDEQKGKDKQLTLITCDDYNEETGVWETRKIFVATEVKLE
HHHHHH.

In the example of the present disclosure, the enzyme ligation preferably includes: mixing the nanodisc with the TMD containing Proxyl site-specifically labeled hCD137, the ECD peptide segment of hCD137, and the Sortase A, and subjecting a resulting mixture to enzyme ligation to obtain the Proxyl site-specifically labeled hCD137 nanodisc. The nanodisc with the TMD containing Proxyl site-specifically labeled hCD137 and the ECD peptide segment of hCD137 are at a molar ratio of (1-4):(1-8), more preferably 1:8, 1:4, 1:2, 4:1, 3:1, or 2:1. The enzyme ligation is preferably conducted at room temperature for 4 h. The Sortase A has a final concentration of preferably 2.5 μM.

In summary, the site-specifically labeled TMD and an ECD are expressed separately, and the TMD is reconstituted into a nanodisc to provide a near-native lipid bilayer environment, and then ligated to the ECD, resulting in a site-specifically labeled full-length transmembrane protein nanodisc. Compared with a traditional preparation method of the transmembrane protein that directly expresses an entire antigen together and conducts purification using detergents, the preparation method of the present disclosure can not only achieve site-specific labeling of the transmembrane protein, but also improve an expression level of transmembrane protein and make the transmembrane protein relatively easier to extract.

Based on the above advantages, the present disclosure further provides use of the site-specifically labeled transmembrane protein nanodisc in antibody screening and/or verification, more preferably antibody screening and verification.

In order to further illustrate the present disclosure, the technical solutions provided by the present disclosure are described in detail below in connection with accompanying drawings and examples, but these examples should not be understood as limiting the claimed scope of the present disclosure.

In order to label transmembrane antigens at a specific site, a cysteine mutation was introduced at any position of TMD for site-specific conjugating. At the same time, an enzyme ligation site (such as a ligation site G of Sortase A) was added to the amino terminus of TMD, and its gene sequence was optimized to the codons preferred by E. coli and then subcloned into an expression plasmid.

### Example 1 Expression and Purification of TMD of Transmembrane Protein

Taking hCD137 TMD as an example, the TMD amino acid sequence containing the enzyme ligation site G of Sortase A and cysteine mutation was: GCIISFFLALTSTALLFLLFFLTLRFSVVKRGRKKLLYIFKQP (SEQ ID NO: 6), a nucleotide sequence encoding the amino acid sequence shown in SEQ ID NO: 1 was 5′-GGCTGTATTATATCATTTTTCCTGGCGCTGACCTCCACCGCTCTGCTGTTCC TCTTGTTTTTCTTGACGCTGCGTTTTAGCGTTGTGAAGCGCGGTCGTAAAAAG CTGTTATACATCTTTAAACAGCCG-3′ (SEQ ID NO: 7). The nucleotide sequence shown in SEQ ID NO: 1 was inserted between the Hind III and BamH I restriction sites of pMM-LR6 to obtain an hCD137 TMD expression vector.

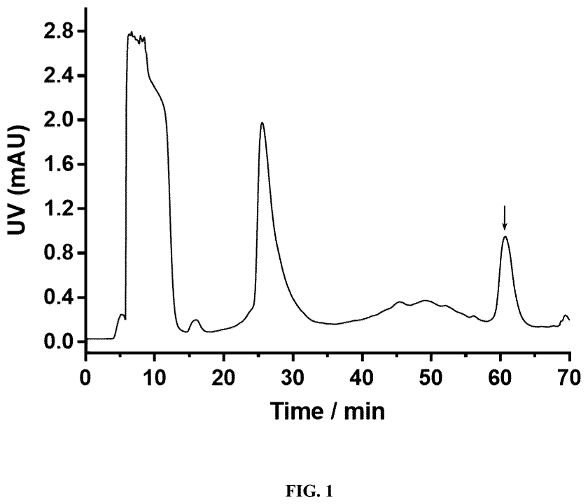

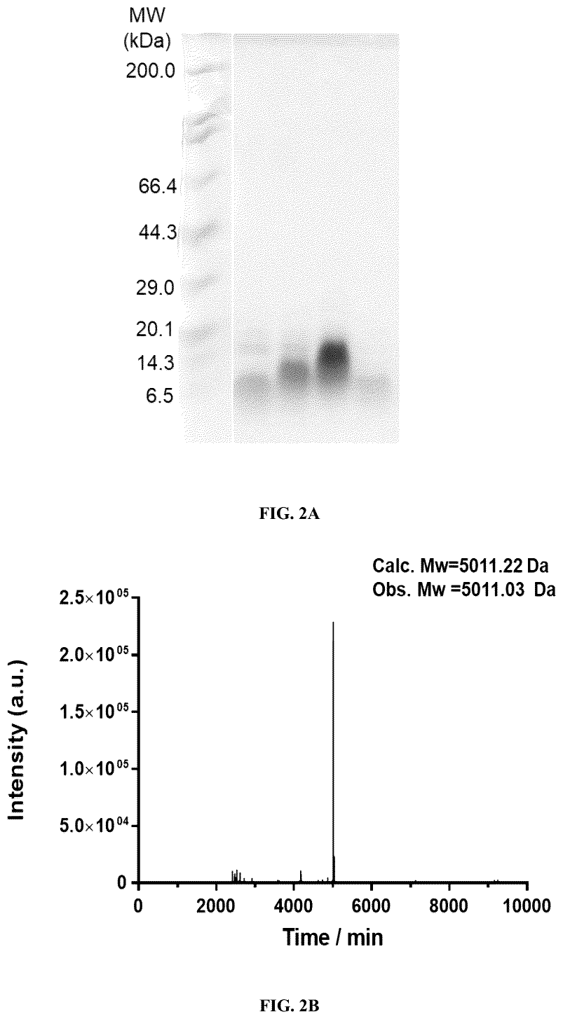

1 μL aliquot of the hCD137 TMD expression vector was transformed into E. coli BL21 (DE3). A single transformed clone was then cultured in LB medium containing 100 μg/mL kanamycin at 37° C. until the optical density at 600 nm (OD₆₀₀) reached 0.6-0.8. At this point, IPTG was added to induce protein expression, and the culture was continued at 18° C. for 18 h. Following expression, the bacterial cells were harvested by centrifugation and the pellet was then resuspended in a Tris buffer (20 mM Tris-HCl, 150 mM NaCl, pH 7.4). Cell disruption was achieved through ultrasonic treatment. Subsequent centrifugation at 12,000 rpm for 30 min, and a resulting supernatant was discarded. The pellet was then resuspended in a guanidine hydrochloride buffer (6 M guanidine hydrochloride, 50 mM Tris, 200 mM NaCl, pH 8.0) and stirred magnetically overnight. Next day, after another centrifugation at 12,000 rpm for 30 minutes, the clear supernatant was collected and subjected to His tag affinity chromatography. The column was washed with the same guanidine hydrochloride buffer, followed by a urea buffer (8 M urea), and finally deionized water. The TMD fusion protein bound to the column was eluted with 90% formic acid. CNBr was added to facilitate cleavage under nitrogen gas. Following cleavage, the reaction mixture was dialyzed against deionized water to remove CNBr and the resulting TMD peptide segment was vacuum freeze-dried. The dry product was then dissolved in a mixture of 1 mL of DMSO and 0.5 mL 90% formic acid, filtered through a 0.8 μm nylon filter, and subjected to high-performance liquid chromatography (HPLC, using Venusil XBP C18 (2)) to allow further isolation and purification. The separation was achieved by gradient elution, where a buffer A was deionized water containing 0.1% TFA, and a buffer B was 30% acetonitrile containing 0.1% TFA+70% isopropyl alcohol. Gradient elution was conducted with 35% to 100% of the buffer B, and peaks of TrpLE, TrpLE-TMD, and TMD emerged successively, as shown in FIG. 1. Tubes containing TMD were collected and lyophilized to obtain hCD137 TMD powder with cysteine, which was recorded as cys-TMD powder, and its purity and molecular weight were characterized by SDS-PAGE gel and mass spectrometry. The results were shown in FIG. 2A-2B.

As shown in FIG. 2A-2B, the SDS-PAGE gel indicated that a main component in the sample was TMD; the mass spectrometry results showed that a molecular weight of the sample was almost consistent with the theoretical molecular weight, such that the target product of this example was obtained.

### Example 2: Site-Specific Conjugation of TMD

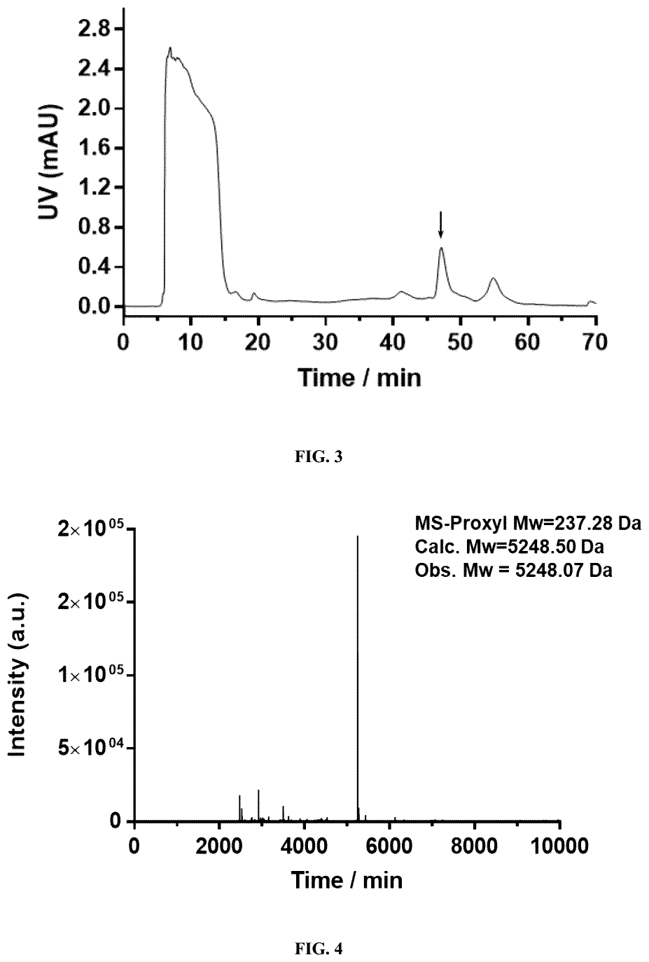

In this example, the conjugation of hCD137 TMD with cysteine (Cys-TMD) and 3-maleimide-2,2,5,5-tetramethylpyrrolidine-1-oxyl radical (3-Maleimido-Proxyl) is demonstrated. The Cys-TMD powder, prepared as described in Example 1, was dissolved in HEPES reaction buffer. To initiate the conjugation reaction, a 1 to 2 molar excess of tris(2-carboxyethyl) phosphine (TCEP) and 3-Maleimido-Proxyl were added to the solution. The components were mixed thoroughly at a molar ratio of 1:2:2 (Cys-TMD:TCEP: 3-Maleimido-Proxyl) and allowed to react at room temperature for 4 h. After blowing dry with N₂, the Proxyl-TMD was purified according to the HPLC purification method in Example 1. Free Proxyl and Proxyl-TMD peaked out successively, as shown in FIG. 3. The Proxyl-TMD was collected and lyophilized to obtain a Proxyl-TMD powder.

The ligation efficiency and purity of hCD137 TMD introduced with cysteine and 3-Maleimido-Proxyl were characterized by mass spectrometry, and the results were shown in FIG. 4.

As shown in FIG. 4, a molecular weight of the product was consistent with the molecular weight of hCD137 TMD-Proxyl, indicating that the product peak in this example was obtained, with a ligation efficiency of 100%.

### Example 3: Preparation of Receptor TMD Nanodisc with Site-Specific Labeling

A Strep tag was added to the carboxyl terminus of membrane scaffold protein (MSP) to facilitate protein purification. The modified MSP, labeled with the Strep tag, exhibited an amino acid sequence shown in SEQ ID NO: 3, specifically:

MGSSTFSKLREQLGPVTQEFWDNLEKETEGLRQEMSKDLEEVKAKVQPYL
DDFQKKWQEEMELYRQKVEPLRAELQEGARQKLHELQEKLSPLGEEMRDR
ARAHVDALRTHLAPYSDELRQRLAARLEALKENGGARLAEYHAKATEHLS
TLSEKAKPALEDLRQGLLPVLESFKVSFLSALEEYTKKLNTQGGGGSSSA
WSHPQFEK.

The gene sequence encoding MSP was optimized to the codons preferred by E. coli, where an optimized nucleotide sequence was shown in SEQ ID NO:8, specifically 5′-ATGGGCAGCAGCACCTTTAGCAAACTGCGTGAACAGCTGGGCCCGGTGAC CCAGGAATTTTGGGATAACCTGGAAAAAGAAACCGAAGGCCTGCGTCAGGA AATGAGCAAAGATCTGGAAGAGGTGAAAGCGAAAGTGCAGCCGTATCTGGA TGACTTTCAGAAAAAATGGCAGGAAGAGATGGAACTGTATCGTCAGAAAGTG GAACCGCTGCGTGCGGAACTGCAGGAAGGCGCGCGTCAGAAACTGCATGAA CTGCAGGAAAAACTGAGCCCGCTGGGCGAAGAGATGCGTGATCGTGCGCGTG CGCATGTGGATGCGCTGCGTACCCATCTGGCGCCGTATAGCGATGAACTGCG TCAGCGTCTGGCGGCCCGTCTGGAAGCGCTGAAAGAAAACGGCGGTGCGCGT CTGGCGGAATATCATGCGAAAGCGACCGAACATCTGAGCACCCTGAGCGAA AAAGCGAAACCGGCGCTGGAAGATCTGCGTCAGGGCCTGCTGCCGGTGCTGG AAAGCTTTAAAGTGAGCTTTCTGAGCGCGCTGGAAGAGTATACCAAAAAACT GAACACCCAGGGCGGCGGCGGCAGCAGCAGCGCGTGGTCGCATCCACAGTT CGAGAAATAA-3′, subcloned into a pET-28b (+) plasmid, and then inserted between the Nco I and Sac I restriction sites to obtain the MSP expression vector.

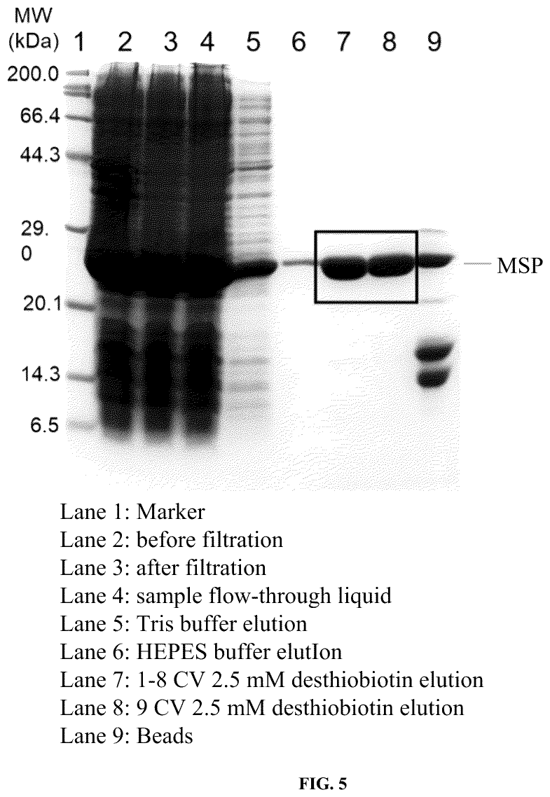

1 μL of the constructed MSP expression vector was transformed into E. coli BL21 (DE3). A single transformed BL21 (DE3) clone was inoculated into LB medium containing 100 μg/mL kanamycin and incubated at 37° C. until OD₆₀₀ reached 0.6-0.8. Expression was then induced by adding IPTG to a final concentration of 0.15 mM. and culturing continued at 37° C. for 4 h. The expressed bacterial cells were collected by centrifugation, resuspended in Tris buffer (20 mM Tris-HCl, 150 mM NaCl, pH 7.4), and disrupted by ultrasonic treatment. After centrifugation at 12,000 rpm for 30 min, the supernatant was collected. MSP was then purified using Streptin affinity column. Impurities were washed away with Tris buffer (20 mM Tris-HCl, 150 mM NaCl, pH 7.4) and HEPES buffer (20 mM HEPES, 75 mM NaCl, pH 7.4). The bound MSP was eluted with HEPES buffer (20 mM HEPES, 75 mM NaCl, pH 7.4) containing 2.5 mM desthiobiotin to obtain the MSP protein. The results were shown in FIG. 5.

As shown in FIG. 5, bands containing only MSP protein were observed in lanes 7 and 8, indicating that the MSP protein was of relatively high purity.

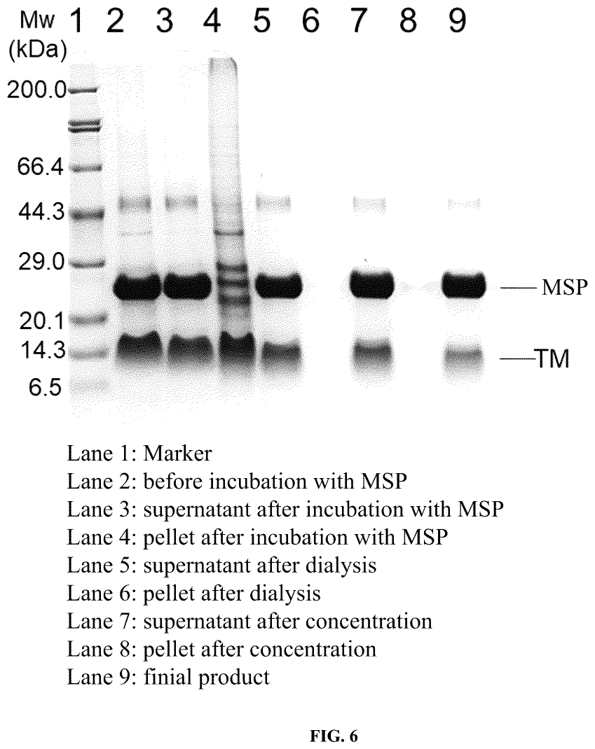

The freeze-dried Proxyl-TMD powder from Example 2 was initially dissolved in urea. Subsequently, DMPC and DHPC were added to achieve a TMD:DMPC:DHPC ratio of 1:35:140. This mixture was blow-dried with N₂ and then lyophilized overnight. The following day, the TMD was re-dissolved in urea buffer (8 M urea, 20 mM HEPES, 75 mM NaCl, pH 7.4), and the urea was removed by dialysis with HEPES buffer (20 mM HEPES, 75 mM NaCl, pH 7.4) to facilitate the reconstitution of bicelle. Following this, MSP protein was added to the bicelle mixture, and dialysis was continued to form the Proxyl-TMD nanodisc, as shown in FIG. 6.

### Example 4: Preparation of Receptor Extracellular Domain (ECD)

A His tag was added between the signal peptide and the amino terminus of ECD for protein purification purpose, and an enzyme ligation site (such as the ligation site LPETGG of Sortase A) was introduced at the carboxyl terminus of ECD. The modified hCD137 ECD, containing both the His tag and the enzyme ligation site of Sortase A, has its amino acid sequence shown in SEQ ID NO: 9, specifically:

HHHHHHLQDPCSNCPAGTFCDNNRNQICSPCPPNSFSSAGGQRTCDICRQ
CKGVFRTRKECSSTSNAECDCTPGFHCLGAGCSMCEQDCKQGQELTKKGC
KDCCFGTFNDQKRGICRPWTNCSLDGKSVLVNGTKERDVVCGPSPADLSP
GASSVTPPAPAREPGHSPQLPETGG.

A gene sequence of hCD137 ECD was optimized to the codons preferred by HEK 293, as shown in SEQ ID NO: 10, specifically 5′-CACCACCACCACCACCACCTGCAGGACCCCTGCTCTAACTGTCCTGCCGGC ACCTTCTGCGATAACAATCGGAACCAGATCTGCTCCCCATGTCCCCCTAATTC TTTTAGCTCCGCCGGCGGCCAGAGGACATGCGATATCTGTCGCCAGTGCAAG GGCGTGTTCCGGACCAGAAAGGAGTGTTCTAGCACAAGCAACGCCGAGTGCG ACTGTACCCCTGGATTTCACTGCCTGGGAGCAGGATGTTCCATGTGCGAGCA GGATTGTAAGCAGGGCCAGGAGCTGACCAAGAAGGGCTGCAAGGACTGCTG TTTCGGCACCTTCAACGATCAGAAGCGGGGCATCTGTAGACCATGGACCAAC TGCAGCCTGGACGGCAAGTCCGTGCTGGTGAATGGCACAAAGGAGAGGGAC GTGGTGTGCGGACCTTCTCCAGCAGATCTGAGCCCAGGAGCATCCTCTGTGA CACCACCAGCACCAGCAAGAGAGCCTGGACACTCCCCACAGCTGCCTGAGAC AGGCGGC-3′, subcloned into a pcDNA3.4 (+) plasmid, and then inserted between the EcoR I and Hind III restriction sites to obtain the hCD137 ECD expression vector.

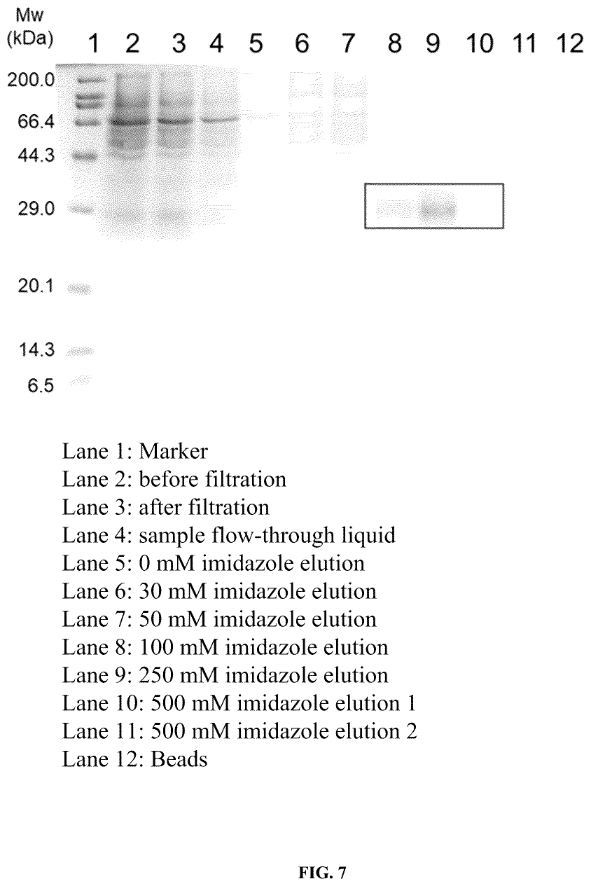

The constructed hCD137 ECD expression vector was transfected into HEK 293F cells, which were maintained at a viability rate of >95% and passaged to a density of 1×10⁶ cells/mL. For transfection, 1 μg plasmid and 3 μg PEI per 1×10⁶ cells were mixed in Opti-MEM, allowed to stand at room temperature for 20 min, and then added dropwise to the cell culture. The cells were then incubated in an incubator for 7 days. On day 7, the culture supernatant was collected by centrifugation for purification. The ECD in the culture supernatant was purified using a His tag affinity column. After loading, the column was washed sequentially with Tris buffer (20 mM Tris-HCl, 150 mM NaCl, 10 μM TCEP, pH 7.4) containing 0 mM, 30 mM, and 50 mM imidazole to remove impurities, monitoring the absorbance at 280 nm (A280). The ECD bound to the column was then eluted with Tris buffer (20 mM Tris-HCl, 150 mM NaCl, 10 μM TCEP, pH=7.4) containing 250 mM and 500 mM imidazole, respectively, yielding higher-purity hCD137 ECD, as shown in FIG. 7.

### Example 5: Preparation of Transmembrane Site-Specifically Labeled Full-Length Receptor hCD137 Nanodisc

A His tag was added to the carboxyl terminus of Sortase A for protein purification. The Sortase A with His tag had an amino acid sequence shown in SEQ ID NO: 5, specifically:

MQAKPQIPKDKSKVAGYIEIPDADIKEPVYPGPATREQLNRGVSFAEENE
SLDDQNISIAGHTFIDRPNYQFTNLKAAKKGSMVYFKVGNETRKYKMTSI
RNVKPTAVEVLDEQKGKDKQLTLITCDDYNEETGVWETRKIFVATEVKLE
HHHHHH.

A gene sequence of Sortase A was optimized to the codons preferred by E. coli, as shown in SEQ ID NO: 11, specifically 5′-ATGCAAGCTAAACCTCAAATTCCGAAAGATAAATCAAAAGTGGCAGGCTA TATTGAAATTCCAGATGCTGATATTAAAGAACCAGTATATCCAGGACCAGCA ACACGCGAACAATTAAATAGAGGTGTAAGCTTTGCAGAAGAAAATGAATCAC TAGATGATCAAAATATTTCAATTGCAGGACACACTTTCATTGACCGTCCGAAC TATCAATTTACAAATCTTAAAGCAGCCAAAAAAGGTAGTATGGTGTACTTTA AAGTTGGTAATGAAACACGTAAGTATAAAATGACAAGTATAAGAAACGTTAA GCCAACAGCTGTAGAAGTTCTAGATGAACAAAAAGGTAAAGATAAACAATT AACATTAATTACTTGTGATGATTACAATGAAGAGACAGGCGTTTGGGAAACA CGTAAAATCTTTGTAGCTACAGAAGTCAAACTCGAGCACCACCACCACCACC AC-3′, subcloned into a pET-28b (+) plasmid, and then inserted between the Nco I and Xho I restriction sites to obtain the Sortase A expression vector.

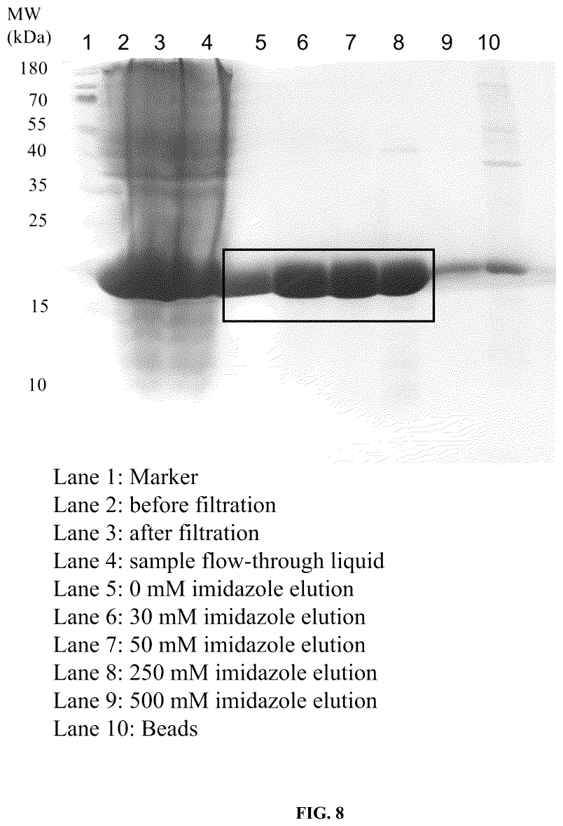

1 μL of the constructed Sortase A expression vector was transformed into E. coli BL21 (DE3). A single transformed BL21 (DE3) clone was inoculated into LB medium containing 100 μg/mL kanamycin and incubated at 37° C. until OD 600 reached 0.6-0.8. IPTG was then added to a final concentration of 1 mM was to induce protein expression, and culturing was continued at 18° C. for 18 h. The cells were collected by centrifugation and then resuspended in a Tris buffer (including 20 mM Tris-HCl, 150 mM NaCl, pH 7.4). The cells were lysed using ultrasonication, followed by centrifugation at 12,000 rpm for 30 min to collect supernatant. Sortase A in the supernatant was purified using a His tag affinity column. After the supernatant passed through the column, the column was washed with Tris buffer (20 mM Tris-HCl, 150 mM NaCl, pH=7.4) containing 0 mM, 30 mM, and 50 mM imidazole separately until impurity proteins did not flow out (a flow-through liquid was collected to detect an A280 absorption of the flow-through liquid with UV until A280 stabilized near a certain value). The Sortase A bound the column was eluted with Tris buffer (20 mM Tris-HCl, 150 mM NaCl, pH=7.4) containing 250 mM and 500 mM imidazole separately, to finally obtain higher-purity Sortase A, as shown in FIG. 8.

The carboxyl terminus of hCD137 ECD was engineered to include the sequence LPETGG, while the amino terminus of TMD was modified to start with a glycine residue (G), enabling Sortase-mediated enzymatic ligation. The hCD137 ECD and TMD-containing nanodisc were mixed at a molar ratio of 1:4, to which Sortase was added. The mixture was incubated at room temperature for 4 h. Since ECD had a His tag, while TMD-containing nanodisc did not, the ligated hCD137 ECD-TM could be obtained through His-tag pulldown, thereby removing the un-ligated TMD-containing nanodisc.

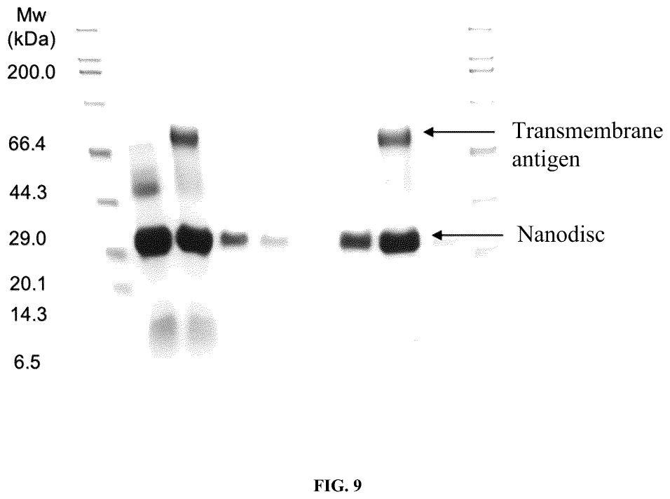

An enzyme ligation product was purified using a His tag affinity column. Unbound materials, including any nanodisc not ligated to ECD, were washed away with HEPES buffer (20 mM HEPES, 75 mM NaCl, 10 μM TCEP, pH 7.4) containing 50 mM imidazole. The ECD-TM nanodisc bound to the column was eluted using 500 mM imidazole, and yielding a higher-purity hCD137 ECD-TM nanodisc, as shown in FIG. 9.

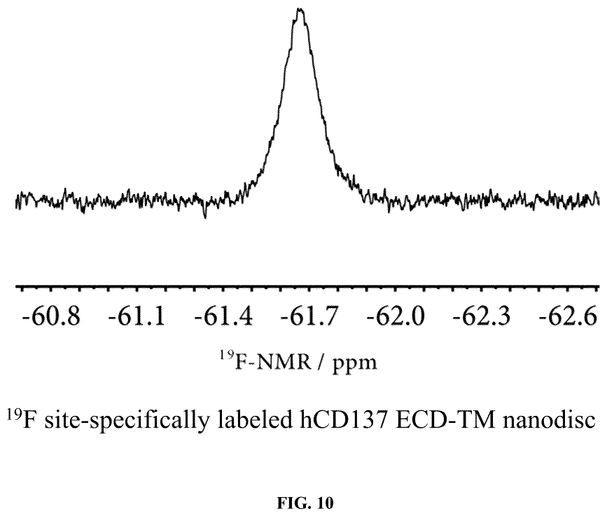

To demonstrate the applicability of this method for the site-specific labeling of various chemical tags, a similar approach was employed to attach trifluoromethyl-containing compounds in the present disclosure. Both bicelle and nanodisc were utilized in this context. The site-specific labeling of the hCD137 transmembrane antigen within nanodisc was confirmed uysing ¹⁹F-solution NMR, as shown in FIG. 10.

### Use Example 1: Functional Verification of Transmembrane Antigen hCD137 with Site-Specifically Labeling

A preparation method of anti-hCD137 nanobody included the following steps:

A His tag and TEV cleavage site were added to the amino terminus of the nanobody to facilitate protein purification. The sequence was shown in SEQ ID NO: 5, specifically:

MHHHHHHENLYFQSEVQLLESGGGEVQPGGSLRLSCAASGFSFSINAMGW
YRQAPGKRREFVAAIESGRNTVYAESVKGRFTISRDNAKNTVYLQMSSLR
AEDTAVYYCGLLKGNRVVSPSVAYWGQGTLVTVKP.

The gene sequence of anti-hCD137 nanobody was optimized to the codons preferred by E. coli, as shown in SEQ ID NO: 12, specifically 5′-ATGCACCACCACCACCACCACGAAAACCTGTACTTTCAGAGCGAGGTGCA ACTGCTGGAAAGCGGTGGCGGTGAGGTTCAACCGGGCGGTAGCCTGCGTCTG AGCTGCGCGGCGAGCGGTTTCAGCTTTAGCATCAACGCGATGGGCTGGTATC GTCAAGCGCCGGGCAAGCGTCGTGAATTCGTGGCGGCGATCGAGAGCGGCC GTAACACCGTGTACGCGGAAAGCGTTAAGGGTCGTTTTACCATTAGCCGTGA CAACGCGAAAAACACCGTGTATCTGCAGATGAGCAGCCTGCGTGCGGAGGAT ACCGCGGTTTACTATTGCGGCCTGCTGAAGGGTAACCGTGTGGTTAGCCCGA GCGTTGCGTACTGGGGCCAAGGTACCCTGGTGACCGTTAAACCG-3′, subcloned into a pET-28b (+) plasmid, and then inserted between the Nco I and Xho I restriction sites to obtain the anti-hCD137 nanobody expression vector.

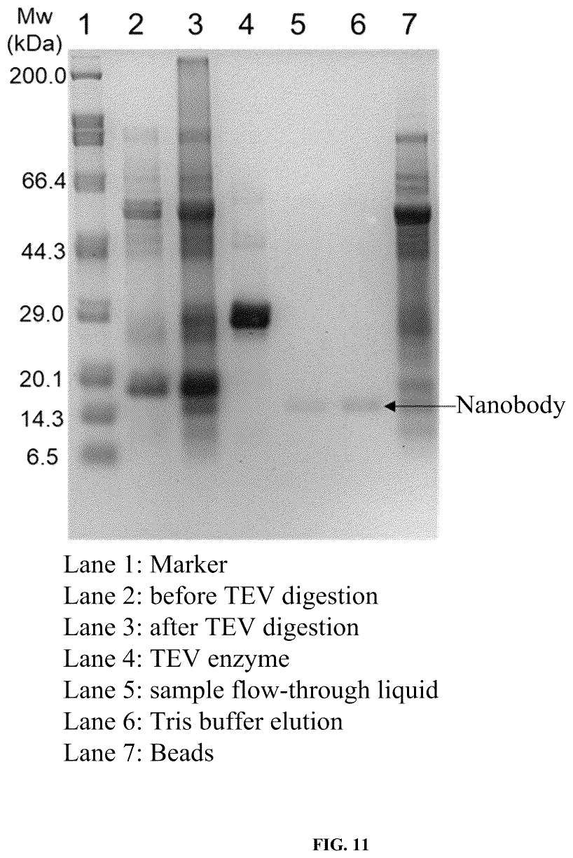

1 μL of the constructed anti-hCD137 nanobody expression vector was transformed into E. coli T7 Shuffle. A single colony was cultured in LB medium containing 100 μg/mL kanamycin at 37° C. until OD₆₀₀ reached 0.6-0.8. IPTG was then added to a final concentration of 0.15 mM to induce protein expression, and culturing was continued at 37° C. for 4 h. The cells were collected by centrifugation, resuspended in a Tris buffer (including 20 mM Tris-HCl, 150 mM NaCl, pH 7.4), and lysed by ultrasonication. The lysate was centrifuged at 12,000 rpm for 30 min, and a resulting supernatant was collected. Nanobody in the supernatant was purified using a His tag affinity column. After the supernatant passed through the column, the column was washed with Tris buffer (20 mM Tris-HCl, 150 mM NaCl, pH 7.4) containing 0 mM, 30 mM, and 50 mM imidazole separately until impurity proteins did not flow out (a flow-through liquid was collected to detect an A280 absorption of the flow-through liquid with UV until A280 stabilized near a certain value). The nanobody bound the column was eluted with Tris buffer (20 mM Tris-HCl, 150 mM NaCl, pH=7.4) containing 250 mM and 500 mM imidazole separately. The eluates were dialyzed against 3 L Tris buffer (20 mM Tris-HCl, 150 mM NaCl, pH 7.4) to remove imidazole with a 3 kDa dialysis bag. After the nanobody was concentrated, TEV protease was added at a ratio of 20:1 (nanobody:TEV) and incubation proceeded at room temperature for 2 hours to cleave the His-tag. The reaction mixture was passed through a His-tag affinity column to bind any undigested nanobody and the TEV protease, allowing the collection of the flow-through containing the cleaved nanobody. This resulted in a higher-purity anti-hCD137 nanobody, as shown in FIG. 11.

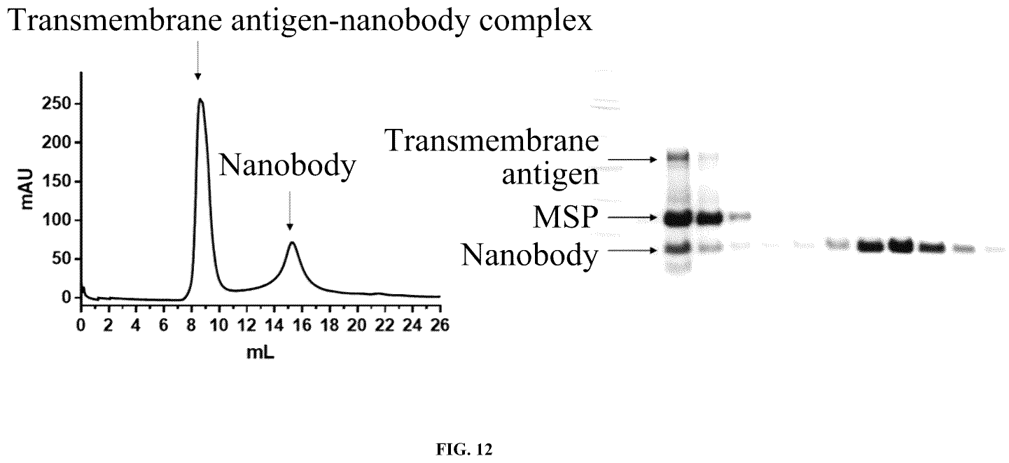

The purified anti-hCD137 nanobody was then mixed with hCD137 ECD-TM-containing nanodisc, prepared as described in Example 5, at a molar ratio of 2:1 (anti-hCD137 nanobody:ECD]). After incubation at room temperature for 2 hours, the mixture was centrifuged, and the supernatant was analyzed using fast protein liquid chromatography (Superdex75 increase 10/300 GL) with a buffer (20 mM HEPES, 75 mM NaCl, 10 μM TCEP, pH=7.4) to assess the binding of the nanobody to the hCD137 antigen. The chromatography revealed distinct peaks in the following order: the hCD137 ECD-TM nanodisc bound to the anti-hCD137 nanobody, free Membrane Scaffold Protein (MSP), hCD137 ECD not ligated to the TMD, and unbound anti-hHER2 nanobody, as shown in FIG. 12

As shown in FIG. 12, the chromatography results confirmed that the anti-hCD137 nanobody co-eluted with the nanodisc containing the hCD137 ECD-TM, demonstrating the binding capability of the full-length transmembrane hCD137 antigen.

### Example 6: Preparation of Site-Specifically Labeled Transmembrane Receptor hHer2 Nanodisc

The methods for preparing site-specifically labeled transmembrane protein nanodisc in Examples 1 to 5 were also applicable to other transmembrane proteins. This example adopted a membrane protein hHER2 as a second case to demonstrate the feasibility and universality of the method for constructing membrane protein nanodisc.

1) hHER2 TMD was prepared according to the method in Example 1, where an amino acid sequence of the TMD, which includes a Sortase A ligation site and a cysteine mutation for conjugation, was shown in SEQ ID NO: 13, specifically:

GGGGGCPAEQRASPLTSIISAVVGILLVVVLGVVFGILIKRRQQKIRKYT
LRRLLQETELVEPLTPSGA.

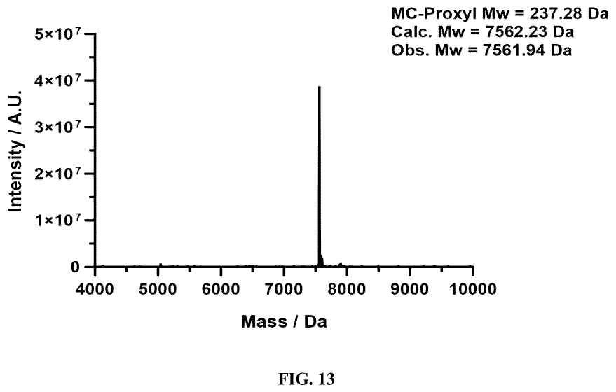

2) The hHER2 TMD prepared in step 1) was conjugated with 3-Maleimido-Proxyl to achieve site-specifically labeling, following the method described in Example 2. The resultant conjugate, Proxyl-hHER2 TMD, as shown in FIG. 13.

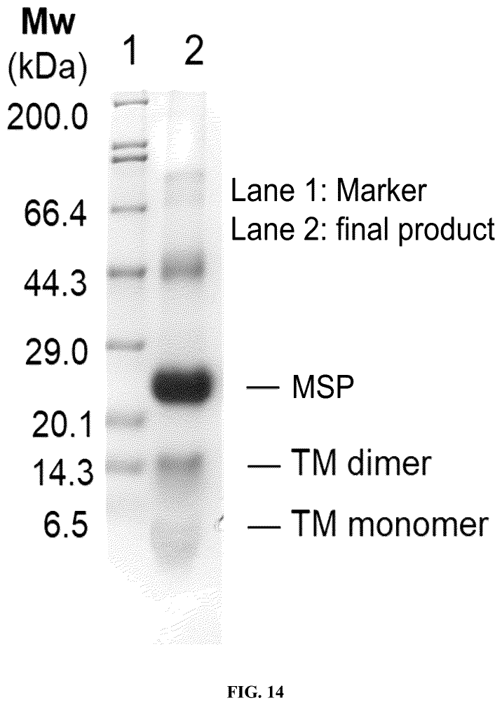

3) The Proxyl-hHER2 TMD nanodisc containing site-specifically labeled site was prepared according to the method in Example 3, and the results were shown in FIG. 14.

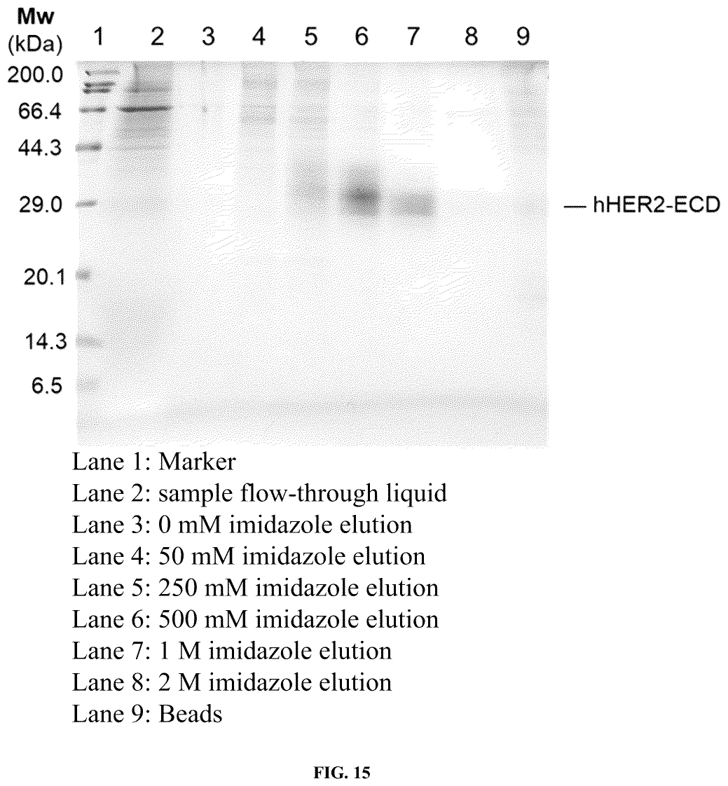

4) Purified hHER2 ECD was expressed according to the method in Example 4, as shown in FIG. 15; the hHER2 ECD with His tag and enzyme ligation site of Sortase A had an amino acid sequence shown in SEQ ID NO: 14, specifically:

HHHHHHHHHHGGGGSHQALLHTANRPEDECVGEGLACHQLCARGHCWGPG
PTQCVNCSQFLRGQECVEECRVLQGLPREYVNARHCLPCHPECQPQNGSV
TCFGPEADQCVACAHYKDPPFCVARCPSGVKPDLSYMPIWKFPDEEGACQ
PCPINCTHSCVDLDDKGLPETGG.

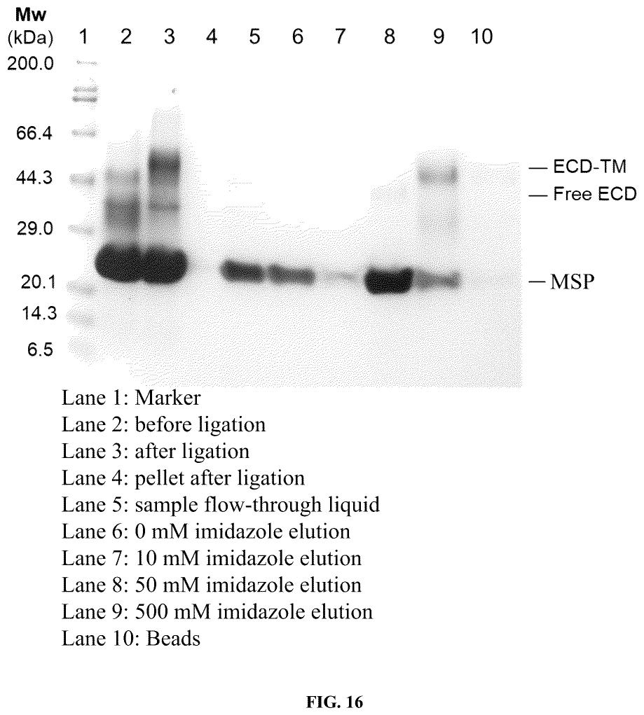

The purified hHER2 ECD and Proxyl-hHER2 TMD nanodisc were ligated using enzyme ligation by Sortase A and purified via His-tag affinity, ultimately yielding the hHER2 ECD-TM nanodisc, as shown in FIG. 16.

### Use Example 2: Functional Verification of Transmembrane Antigen hHer2 with Site-Specifically Labeling

A preparation method of anti-hHER2 nanobody included the following steps:

A His tag and TEV cleavage site were added to the amino terminus of the nanobody for protein purification. A sequence was shown in SEQ ID NO: 15, specifically:

MGSAHHHHHHGGGSGGGSENLYFQSEVQLVESGGGLVQAGGSLRLSCAAS
GITFSINTMGWYRQAPGKQRELVALISSIGDTYYADSVKGRFTISRDNAK
NTVYLQMNSLKPEDTAVYYCKRFRTAAQGTDYWGQGTLVTVSSGSC.

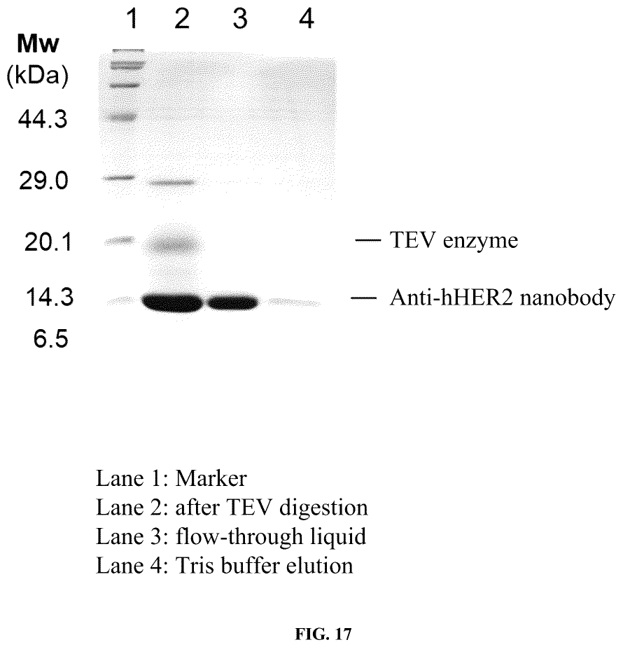

A gene sequence of anti-hHER2 nanobody was optimized to the codons preferred by Saccharomyces, as shown in SEQ ID NO: 16, specifically 5′-ATGGGATCAGCTCACCACCATCACCACCACGGTGGTGGCTCGGGGGGGG CTCGGAGAACCTGTATTTCCAGAGCGAGGTTCAACTGGTGGAGAGTGGTGGC GGCCTGGTGCAAGCGGGTGGTTCCCTGAGACTGAGCTGCGCGGCGAGCGGCA TTACCTTTAGCATCAACACGATGGGTTGGTACCGCCAGGCTCCGGGTAAACA GCGTGAACTTGTCGCACTCATCAGCTCCATCGGCGACACCTATTACGCAGACT CCGTGAAGGGTCGTTTCACCATTAGCCGTGACAACGCGAAAAATACCGTTTA CCTGCAGATGAATAGCTTGAAGCCGGAAGATACTGCGGTTTACTACTGCAAA CGTTTTCGCACCGCTGCCCAAGGTACGGATTATTGGGGTCAGGGTACGTTGGT TACCGTGAGCTCTGGTTCTTGTTAA-3′, subcloned into a pET-28b (+) plasmid, and then inserted between the Nco I and Xhol I restriction sites to obtain the anti-hHER2 nanobody expression vector. Expression and purification were conducted according to the method in Use Example 1 to obtain higher-purity anti-hHER2 nanobody, as shown in FIG. 17.

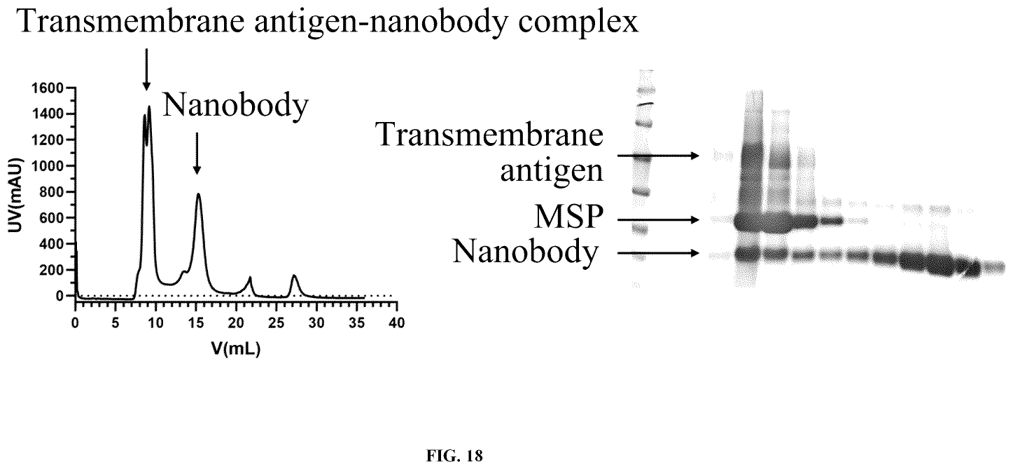

The purified anti-hHER2 nanobody was added to the hHER2 ECD-TM-containing nanodisc prepared in Example 6 at a molar concentration ratio of [anti-hHER2 nanobody]:[ECD]-3:1. This mixture was thoroughly mixed and then incubated at room temperature for 2 h. Following incubation, the mixture was centrifugated at 12,000 rpm for 3 min, and the supernatant was collected. The biological activity of hHER2 ECD-TM nanodisc was assessed using fast protein liquid chromatography (Superdex75 increase 10/300 GL) with a buffer (20 mM HEPES, 75 mM NaCl, 10 μM TCEP, pH=7.4). The chromatography revealed distinct peaks in the following order: the hHER2 ECD-TM nanodisc bound to the anti-hHER2 nanobody, free Membrane Scaffold Protein (MSP), hHER2 ECD not ligated to the TMD, and unbound anti-hHER2 nanobody, as shown in FIG. 18.

As shown in FIG. 18, anti-hHER2 nanobody could co-elute with hHER2 ECD-TM nanodisc, indicating that the transmembrane antigen hHER2 nanodisc showed antibody-binding capabilities.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

1. A preparation method of a site-specifically labeled transmembrane domain (TMD), comprising the following steps:
introducing a cysteine mutation into a TMD gene fragment encoding a transmembrane protein to obtain a cysteine mutation-containing TMD gene fragment;
expressing the cysteine mutation-containing TMD gene fragment to obtain a cysteine mutation site-containing TMD peptide segment; and
conjugating a chemical compound to a cysteine mutation site on the cysteine mutation site-containing TMD peptide segment to obtain the site-specifically labeled TMD.

2. The preparation method according to claim 1, wherein the transmembrane protein comprises a transmembrane protein containing an extracellular domain (ECD) and a TMD.

3. A site-specifically labeled TMD nanodisc, comprising a site-specifically labeled TMD and a nanodisc; wherein the nanodisc is wrapped on or inserted into the site-specifically labeled TMD; and the site-specifically labeled TMD is prepared by the preparation method according to claim 1.

4. The site-specifically labeled TMD nanodisc according to claim 3, wherein the nanodisc comprises a phospholipid nanodisc.

5. A site-specifically labeled transmembrane protein nanodisc, comprising the site-specifically labeled TMD nanodisc according to claim 3 and an ECD.

6. A preparation method of the site-specifically labeled transmembrane protein nanodisc according to claim 5, comprising: ligating an amino terminus of the site-specifically labeled TMD nanodisc to a carboxyl terminus of the ECD to obtain the site-specifically labeled transmembrane protein nanodisc.

7. The preparation method according to claim 6, wherein the ligating is conducted by enzyme ligation or peptide chain ligation.

8. The preparation method according to claim 7, wherein an enzyme for the enzyme ligation is selected from the group consisting of a Sortase and a Butelase.

10. A method for antibody screening and/or verification, comprising using the site-specifically labeled TMD nanodisc according to claim 3.

11. A site-specifically labeled TMD nanodisc, comprising a site-specifically labeled TMD and a nanodisc; wherein the nanodisc is wrapped on or inserted into the site-specifically labeled TMD; and the site-specifically labeled TMD is prepared by the preparation method according to claim 2.

12. The site-specifically labeled TMD nanodisc according to claim 11, wherein the nanodisc comprises a phospholipid nanodisc.

13. A site-specifically labeled transmembrane protein nanodisc, comprising the site-specifically labeled TMD nanodisc according to claim 11 and an ECD.

14. A site-specifically labeled transmembrane protein nanodisc, comprising the site-specifically labeled TMD nanodisc according to claim 4 and an ECD.

15. A site-specifically labeled transmembrane protein nanodisc, comprising the site-specifically labeled TMD nanodisc according to claim 12 and an ECD.

16. A method for antibody screening and/or verification, comprising using the site-specifically labeled TMD nanodisc according to claim 4.

17. A method for antibody screening and/or verification, comprising using the site-specifically labeled transmembrane protein nanodisc according to claim 5.

18. A method for antibody screening and/or verification, comprising using a site-specifically labeled transmembrane protein nanodisc prepared by the preparation method according to claim 7.

19. A method for antibody screening and/or verification, comprising using a site-specifically labeled transmembrane protein nanodisc prepared by the preparation method according to claim 8.
